(19) United States
(12) Patent Application Publication
(10) Pub. No.: US 2021/0045928 A1
(43) Pub. Date: Feb. 18, 2021

(54) BLISTER PROTECTOR CAP

(71) Applicant: David Derohanian, Glendale, CA (US)

(72) Inventor: David Derohanian, Glendale, CA (US)

(21) Appl. No.: 16/992,022

(22) Filed: Aug. 12, 2020

Related U.S. Application Data
(60) Provisional application No. 62/885,772, filed on Aug. 12, 2019.

(51) Int. Cl. A61F 13/06; A61F 13/02

(52) U.S. Cl. CPC A61F 13/063; A61F 2013/00165; A61F 13/0236

(57) ABSTRACT

A blister protector cap incorporates features such as a hemispherical/paraboloid cap dome, which goes over a blister and attaches to the skin using an adhesive compound. The blister protector cap may have air vents located at any part of its surface for ventilation purposes and aiding the skin to heal the blister. The anterior side of the blister protector cap has a cavity in order to cover the blister and protect it from any sharp or rough objects. In an embodiment, some part of the dome is flexible. In such a configuration, an accordion-like wall structure or a corrugated wall structure can be integrated to allow for some flexibility. Furthermore, this flexible part of the dome can also have vent holes or be made of a porous material to allow air and moisture to pass through but block water molecules from passing through to the inside of dome.

### PRIORITY CLAIMS AND CROSS-REFERENCE TO RELATED APPLICATIONS

This application is related to and claims domestic priority benefits, per 35 USC § 119(e), from U.S. Provisional Patent Application Ser. No. 62/885,772 filed on Aug. 12, 2020, the entire contents, of the aforementioned application, are expressly incorporated hereinto by reference.

### BACKGROUND

Currently there are many over-the-counter blister protectors available to the consumer. Most of them are based on a patch or gel cushion system to provide support and padding. All of these designs however suffer from the same flaw of coming into contact with the blister itself and running the risk of agitating the blister and the area of the skin surrounding the blister. Because users of these products do not get full relief from using them, they are disinclined to move around, wear shoes, and participate in regular day-to-day activities until the blister has healed. Naturally, this process can take multiple days or even weeks to resolve. In the interim, the patient cannot wait that long for the skin to heal, at the same time ensuring that the blister is not agitated in any way.

Therefore, in this application, a blister protector device is proposed which works synergistically with the skin in allowing the blister to heal. The proposal is based on the concept of shielding the blister from perturbations caused by normal human activities. The notable concern is protecting the blister from being rubbed against a rough object which would cause it to be agitated, and thus prolonging the healing process.

### SUMMARY

The present disclosure relates to, but is not limited to, a blister protector cap apparatus which has a hemispheroid dome or a paraboloid dome with an empty cavity on one side. The hemispheroid dome or the paraboloid dome having a base with a larger diameter than the diameter of the longest cross-section of either the hemispheroid dome or the paraboloid dome. The base having an adhesive compound incorporated on one surface portion of the base.

In another embodiment, the blister protector cap apparatus has a hemispheroid dome or a paraboloid dome with an empty cavity on one side. The hemispheroid dome or the paraboloid dome having a base with a larger diameter than the diameter of the longest cross-section of either the hemispheroid dome or the paraboloid dome. The base having an adhesive compound incorporated on one surface portion of the base where the adhesive compound extends beyond the bounds of the base radiating outwards away from the base.

In yet another embodiment, a blister protector cap apparatus has a hemispheroid dome or a paraboloid dome with an empty cavity on one side. The hemispheroid dome or the paraboloid dome having a base with a larger diameter than the diameter of the longest cross-section of either the hemispheroid dome or the paraboloid dome, where the hemispheroid dome or the paraboloid dome have air vent holes on the surface. The base having an adhesive compound incorporated on one surface portion of the base.

### BRIEF DESCRIPTION OF THE DRAWINGS

Embodiments in accordance with the present disclosure are shown in the drawings and will be described below with reference to the figures, whereby elements having the same effect have been provided with the same reference numerals. The following is shown:

FIG. 1 shows a side view of one embodiment of the blister protector cap;

FIG. 2 shows a top-down view of one embodiment of the blister protector cap;

FIG. 3 shows an anterior view of one embodiment of the blister protector cap;

FIG. 4A shows a side view of another embodiment of the blister protector cap with an extended adhesive material;

FIG. 4B shows a top-down view of the blister protector cap of FIG. 4A;

FIG. 5 shows a top-down view of another embodiment of the blister protector cap indicating the air vents on the surface of the dome; and

FIG. 6 shows a side view of another embodiment of the blister protector cap where part of the girth of the dome cap is made of an accordion-like or corrugated semi-flexible material and/or fabric.

### DETAILED DESCRIPTION

An embodiment of the blister protector cap device is described herein. However, it will be clear and apparent to one skilled in the art that the blister protector cap device is not limited to the embodiments set forth herein.

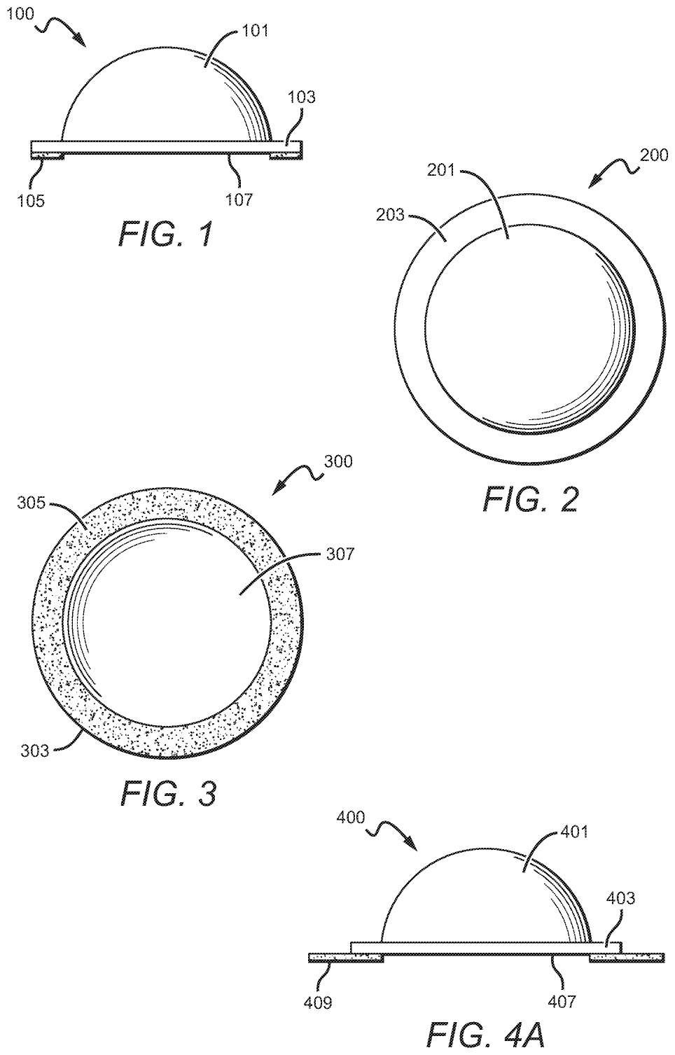

FIG. 1 shows the various components of the blister protector cap device 100. At the top of the device 100, the dome 101 is protruding from the base 103 of the blister protector cap device 100. The dome 101 may be made of a clear plastic, any rigid or semi-rigid material capable of keeping its form when objects come into contact with the blister protector cap device 100. The dome 101 may be a hemispheroid or a paraboloid shape with a certain thickness.

The base 103 is a circular shaped ring surrounding and partially protruding from the base of the dome 101 and may be integrated with the body of the dome 101. The base 103 may have the same thickness of the dome 101 but is not limited to that. On the underside of the base 103, there is a surface area where the adhesive compound 105 can attach or incorporate into the base 103. The surface area—not shown—can extend partially into the underside of the base 103 at some distance from the edge of the base 103. The entire circumference of the base 103 may be laden with this adhesive compound 105. The adhesive compound 105 is used to secure the blister protector cap device 100 on to the patient's skin. The adhesive compound 105 can be made of a compatible adhesive material such as a medical grade adhesive tape or the like.

The anterior side of the blister protector cap device 100 contains a cavity 107. The cavity 107 is large enough in width and height to accommodate various sized blisters. In some embodiments, the cavity 107 size will vary depending on the size of the blister. Thus, the patient has the ability to use a different sized blister protector cap device 100 that vary in cavity 107 size based on their needs.

FIG. 2 shows a top-down view of one embodiment of the blister protector cap device 200. The dome 201 is rigid, as discussed above, and can be made of a transparent or semi-transparent material. The base 203 illustrates that it is slightly bigger in diameter than the base of the dome 201. The base 203 is larger in diameter in order to employ a large enough surface area to allow for attachment or integration of the adhesive compound 105 (not pictured).

FIG. 3 is an anterior view of the blister protector cap device 300. The blister protector cap device 300 has a cavity 307 that is suitable to receive a typical sized blister. Preferably, there is ample clearance in the cavity 307 between the blister and the interior wall of the dome 101. The underside of the base 303 is shown to have the adhesive compound 305 impregnated upon it. The adhesive compound 305 may have a protective layer (not pictured) as a cover. Once that layer is peeled off, the blister protector cap device 300 can be placed unto a patient's skin and pressed down on the dome 101 in order for the adhesive compound 305 to stick unto the patient's skin.

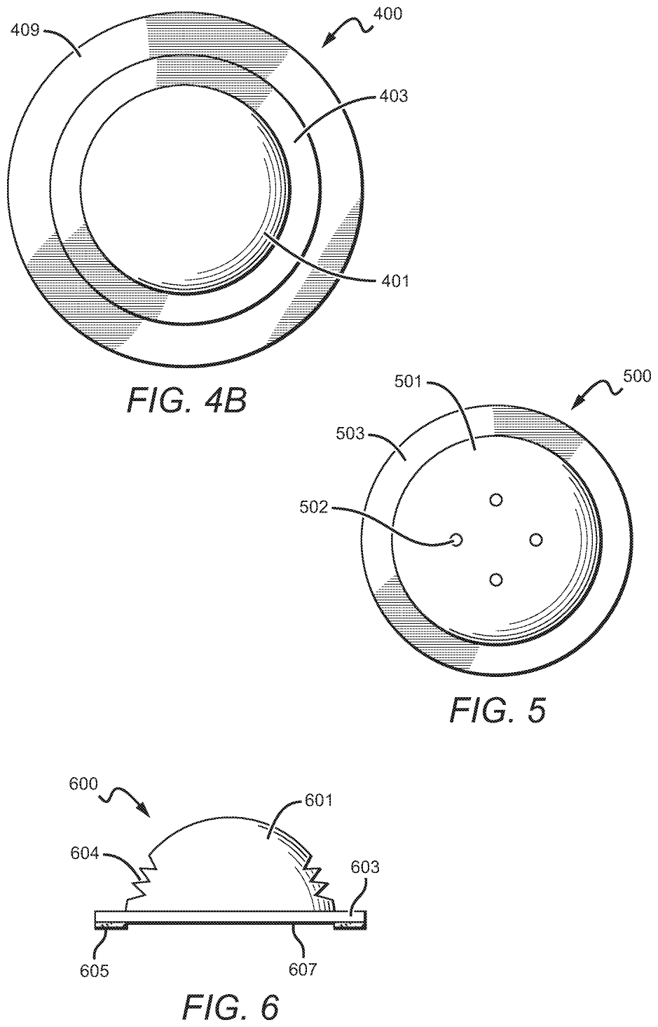

FIG. 4A is a side view of another embodiment of the blister protector cap device 400. In this embodiment, the dome 401 is shown similar to the other embodiments. The base 403 is also shown that is similar to the embodiments discussed above. However, this embodiment has an adhesive material that is larger in surface area than normal. The adhesive skirt 409 expands out in radial fashion to cover a larger surface area of the skin. This is done in order to keep the blister protector cap device 400 in a secure position. In case where the patient comes into contact with a foreign object, with a momentum high enough to knock over the blister protector cap device 400, the adhesive skirt 409 ensures that the blister protector cap device 400 does not go flying off of the skin of the patient. The adhesive skirt 409 may be made of the same material as discussed above or some other material to allow it to retain its shape, be flexible, and bind to the blister protector cap device 400 and/or to the patient's skin. In other words, the adhesive skirt 409 may be made of a flexible fabric, nylon or material which will have the adhesive compound deposited on it. The adhesive skirt 409, and the necessary adhesive incorporated thereon, surrounds the whole perimeter of the base 403. Furthermore, the opening 407 is also shown where the blister would be housed in.

FIG. 4B is a top-down view of the blister protector cap device 400 as discussed above with relation to FIG. 4A. The dome 401 is shown in the center which can be made of the same material as discussed in the other embodiments. The blister protector cap device's 400 base 403 is also shown protruding from the bottom of the dome 401. The adhesive skirt 409 further extends the radius of the blister protector cap device 400 allowing for a better contact with the patient's skin.

FIG. 5 shows a top-down view of another embodiment of the blister protector cap device 500. This embodiment incorporates small air vents 502 in some pattern strewn around on the dome 501. The air vents 502 are intended to allow the skin to “breath” properly and to dissipate any moisture that would otherwise accumulate if the patient is sweating profusely. Having an opening for the blister to dry and the skin to heal itself is sometimes desirable based on the patient's skin characteristics and immune response. Furthermore, the base 503 of the blister protector cap device 500 can be seen in this depiction.

FIG. 6 shows a sideview of another embodiment of the blister protector cap device 600. The dome 601 of this embodiment is not a completely rigid or semirigid hemispheroid or a paraboloid. Part of the dome 601 integrates a flexible membrane 604 that may integrate “accordion-like” folds or a corrugated layer. The flexible membrane 604 can be shaped to have a saw-tooth design or a sinusoidal design in its folds. The flexible membrane 604 is situated some distance from the base 603 to the very apex of the dome 601. The flexible membrane 604 can be of a varying length in that regard. The flexible membrane 604 is intended to allow some movement of the dome 601 such that any object that hits the blister protector cap device 600 may have the impact absorbed by the flexible membrane 604. Furthermore, the membrane 604 provides some comfort for the patient as the blister protector cap device 600 may be applied to a foot that would then be covered by a sock and a shoe. By allowing some flexibility, the blister can both be protected and the patient can wear the blister protector cap device 600 without undue discomfort.

In another variation, the flexible membrane 604 can have air vents present in the dome 601 in order for the blister and the surrounding skin to be able to “breath” and dispel any moisture. Furthermore, the flexible membrane 604 can instead be made of some porous material whereby air and moisture molecules can penetrate but water molecules can be blocked.

In this embodiment, the base 603 has the adhesive compound 605 attached to its rim and the cavity has the opening 607 at the bottom. The adhesive compound 605 can be placed along the whole perimeter of the base 603.

It is contemplated that these embodiments can borrow features of another embodiment or incorporate all the features into one embodiment.

Although the present invention has been described with reference to the preferred embodiment thereof, it is apparent to those skilled in the art that a variety of modifications and changes may be made without departing from the scope of the present invention which is intended to be defined by the appended claims.

What is claimed is:

1. A blister protector cap apparatus, comprising:
a hemispheroid dome or a paraboloid dome with an empty cavity on one side;
the hemispheroid dome or the paraboloid dome having a base with a larger diameter than the diameter of the longest cross-section of either the hemispheroid dome or the paraboloid dome; and
the base having an adhesive compound incorporated on one surface portion of the base.

2. The apparatus of claim 1, wherein:
the adhesive compound extends beyond the bounds of the base radiating outwards away from the base.

3. The apparatus of claim 1, wherein:
the hemispheroid dome or the paraboloid dome have air vent holes on the surface.

4. The apparatus of claim 1, wherein:
the hemispheroid dome or the paraboloid dome have a radial section of the dome integrated with a collapsible semi-rigid section allowing some movement of the top part of the dome inwards into the bottom part of the dome.

5. The apparatus of claim 4, wherein the collapsible semi-rigid section further comprises:
air vent holes on the surface of the collapsible semi-rigid section and/or the collapsible semi-rigid section is made of a porous material impermeable to water but permeable to moisture.

6. The apparatus of claim 4, wherein:
the adhesive compound extends beyond the bounds of the base radiating outwards away from the base.

7. The apparatus of claim 4, wherein:
the hemispheroid dome or the paraboloid dome have air vent holes on the surface.

8. A blister protector cap apparatus, comprising:
a hemispheroid dome or a paraboloid dome with an empty cavity on one side;
the hemispheroid dome or the paraboloid dome having a base with a larger diameter than the diameter of the longest cross-section of either the hemispheroid dome or the paraboloid dome; and
the base having an adhesive compound incorporated on one surface portion of the base where the adhesive compound extends beyond the bounds of the base radiating outwards away from the base.

9. A blister protector cap apparatus, comprising:
a hemispheroid dome or a paraboloid dome with an empty cavity on one side;
the hemispheroid dome or the paraboloid dome having a base with a larger diameter than the diameter of the longest cross-section of either the hemispheroid dome or the paraboloid dome, where the hemispheroid dome or the paraboloid dome have air vent holes on the surface; and
the base having an adhesive compound incorporated on one surface portion of the base.

10. The apparatus of claim 9, wherein:
the hemispheroid dome or the paraboloid dome have a radial section of the dome integrated with a collapsible semi-rigid section allowing some movement of the top part of the dome inwards into the bottom part of the dome.

11. The apparatus of claim 8, wherein:
the hemispheroid dome or the paraboloid dome have a radial section of the dome integrated with a collapsible semi-rigid section allowing some movement of the top part of the dome inwards into the bottom part of the dome.
